(19) United States
(12) Patent Application Publication
(10) Pub. No.: US 2008/0176905 A1
(43) Pub. Date: Jul. 24, 2008

(54) Polymorphic forms of rosiglitazone hydrobromide and processes for preparation thereof

(76) Inventors: Santiago Ini, Haifa (IL); Kobi Chen, Kfar-Saba (IL); Michal Rafilovich, Petach-Tikva (IL)

Correspondence Address: KENYON & KENYON LLP, ONE BROADWAY, NEW YORK, NY, 10004, US

(21) Appl. No.: 12/009,876

(22) Filed: Jan. 22, 2008

Related U.S. Application Data
(60) Provisional application No. 60/897,010, filed on Jan. 22, 2007.
(60) Provisional application No. 60/899,165, filed on Feb. 1, 2007.
(60) Provisional application No. 60/903,199, filed on Feb. 22, 2007.

(51) Int. Cl. A61K 31/4439; C07D 213/02; A61P 3/00

(52) U.S. Cl. 514/342; 546/269.7

(57) ABSTRACT

The present invention presents crystal forms of Rosiglitazone hydrobromide, denominated Form I and Form II, methods of their preparation, as well as pharmaceutical compositions comprising these crystalline forms.

### CROSS REFERENCE TO RELATED APPLICATIONS

The present application claims the benefit of the following U.S. Provisional Patent Application Nos. 60/897,010, filed Jan. 22, 2007; 60/899,165, filed Feb. 1, 2007; and 60/903,199, filed Feb. 22, 2007. The contents of these applications are incorporated herein by reference.

### FIELD OF THE INVENTION

The present invention related to the solid state chemistry of Rosiglitazone hydrobromide, specifically, new crystalline forms of Rosiglitazone hydrobromide salt, denominated Form I and Form II, as well as to methods for their preparation.

### BACKGROUND OF THE INVENTION

Rosiglitazone (5-((4-(2-(methyl-2-pyridinylamino)ethoxy)phenyl)methyl)-2,4-thiazolidinedione is an anti-diabetic drug from the thiazolidinedione class which acts primarily by increasing insulin sensitivity. This molecule has the following structure:

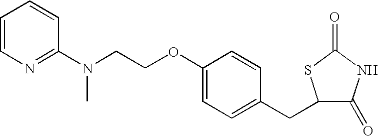

International Patent Application No. WO94/05659, describes salts of Rosiglitazone, including Rosiglitazone maleate. Rosiglitazone maleate is marketed in the U.S. under the trade name: AVANDIA® in 2 mg, 4 mg, and 8 mg tablets. Rosiglitazone maleate and its meleate salt are described in the following U.S. patents, hereby incorporated by reference: U.S. Pat. Nos. 5,002,953; 5,741,803; and 6,288,095.

Rosiglitazone hydrobromide salt is described in International patent Application No. WO 01/94344 (EP 1,296,980). This salt is characterized by IR, Raman, X-Ray powder diffraction, solid state ¹³C-NMR and a melting point. Crystalline forms of this salt are apparently produced by reacting Rosiglitazone or a salt thereof, dispersed or dissolved in a suitable solvent, with a source of hydrogen bromide, following by its recovery. In the examples of WO 01/94344, propan-2-ol and acetone are used.

In addition, U.S. Publication No. 2006/0083784 described amorphous Rosiglitazone hydrobromide.

The present invention relates to the solid state physical properties of Rosiglitazone hydrobromide. These properties can be influenced by controlling the conditions under which Rosiglitazone hydrobromide is obtained in solid form. Solid state physical properties include, for example, the flow-ability of the milled solid. Flow-ability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular polymorphic form of a substance. The polymorphic form may give rise to thermal behavior different from that of the amorphous material or another polymorphic form. Thermal behavior is measured in the laboratory by such techniques as capillary melting point, thermogravimetric analysis (TGA) and differential scanning calorimetric (DSC) and can be used to distinguish some polymorphic forms from others. A particular polymorphic form may also give rise to distinct spectroscopic properties that may be detectable by powder X-ray crystallography, solid state ¹³C NMR spectrometry and infrared spectrometry.

One of the most important physical properties of a pharmaceutical compound, which can form polymorphs or solvates, is its solubility in aqueous solution, particularly the solubility in gastric juices of a patient. Other important properties relate to the ease of processing the form into pharmaceutical dosages, as the tendency of a powdered or granulated form to flow and the surface properties that determine whether crystals of the form will adhere to each other when compacted into a tablet.

The discovery of new polymorphic forms and solvates of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic.

Thus, there is a need in the art for new polymorphs of Rosiglitazone hydrobromide and processes for the preparation of Rosiglitazone hydrobromide forms.

### SUMMARY OF THE INVENTION

In one embodiment, the present invention provides Rosiglitazone hydrobromide having a water content of between 1.0 and 4.1% by weight. Preferably, the Rosiglitazone hydrobromide is crystalline.

In another embodiment, the present invention provides crystalline Rosiglitazone hydrobromide quarter-hydrate.

In yet another embodiment, the present invention provides crystalline Rosiglitazone hydrobromide hemihydrate.

In yet another embodiment, the present invention provides crystalline Rosiglitazone hydrobromide monohydrate.

In one embodiment, the present invention provides a crystalline form of Rosiglitazone hydrobromide, characterized by at least one of data selected from the group consisting of: a powder XRD pattern having peaks at about 15.2, 17.8, 22.9 and 24.5±0.2 degrees two-theta; a solid-state ¹³C-NMR spectrum having chemical shift resonances at about 169.7, 156.5, 135.7, 118.3 and 115.6±0.2 ppm; a solid-state ¹³C NMR spectrum having chemical shift differences between the lowest ppm resonance in the chemical shift area of 100 to 180 ppm and another in the chemical shift area of 100 to 180 ppm of about 59.5, 46.3, 25.5, 8.1 and 5.4±0.1 ppm; an X-ray diffractogram substantially as depicted in FIG. 1; and a solid-state ¹³C-NMR substantially as depicted in FIG. 2 or 3.

The present invention further encompasses a process for preparing the above Rosiglitazone hydrobromide by a process comprising; providing a mixture of Rosiglitazone and a solvent selected from the list consisting of methyl ethyl ketone, C₃₋₇ esters, water and mixture thereof; and admixing the mixture with a source of hydrobromide; and cooling, preferably to a temperature of about 0° C. to about 60° C. to obtain a precipitate.

In another embodiment, the present invention provides a crystalline form of Rosiglitazone hydrobromide, characterized by a powder XRD pattern having peaks at about 4.6, 9.3, 15.3, 16.3 and 18.3±0.2 degrees two-theta.

The present invention further encompasses a process for preparing the above Rosiglitazone hydrobromide characterized by a powder XRD pattern with peaks at about 4.6, 9.3, 15.3, 16.3 and 18.3±0.2 degrees two-theta by a process comprising; providing a mixture of Rosiglitazone and ethylacetate; admixing the mixture with a source of hydrobromide; cooling preferably to a temperature of about 0 to about 60° C. to obtain an oil; and admixing the said oil with a C₁-C₅ alkyl alcohol to obtain a precipitate.

The present invention comprises a pharmaceutical composition comprising the Rosiglitazone hydrobromide crystalline forms described above or mixtures thereof and at least one pharmaceutically acceptable excipient.

The present invention comprises a pharmaceutical composition comprising the Rosiglitazone hydrobromide crystalline forms described above made by the processes of the present invention, and at least one pharmaceutically acceptable excipient.

The present invention further encompasses a process for preparing a pharmaceutical formulation comprising combining the Rosiglitazone hydrobromide crystalline forms described above with at least one pharmaceutically acceptable excipient.

The present invention further encompasses a process for preparing a pharmaceutical formulation comprising combining the Rosiglitazone hydrobromide crystalline forms described above made by the processes of the present invention, and at least one pharmaceutically acceptable excipient.

The present invention further encompasses the use of the Rosiglitazone hydrobromide crystalline forms described above for the manufacture of a pharmaceutical composition.

The present invention further encompasses the use of the Rosiglitazone hydrobromide crystalline forms described above made by the processes of the invention, for the manufacture of a pharmaceutical composition.

### BRIEF DESCRIPTION OF THE FIGURES

FIG. 1 illustrates a powder X-ray diffraction pattern for Rosiglitazone hydrobromide Form I.

FIG. 2 illustrates a powder solid-state ¹³C-NMR spectrum for Rosiglitazone hydrobromide Form I, as prepared by Example 2.

FIG. 3 illustrates a solid-state ¹³C-NMR spectrum Rosiglitazone hydrobromide Form I, as prepared by Example 2 in the chemical shift area of 100 to 180 ppm.

FIG. 4 illustrates a powder X-ray diffraction pattern for Rosiglitazone hydrobromide Form II.

### DETAILED DESCRIPTION

The present invention provides novel crystalline forms of Rosiglitazone hydrobromide.

As used herein, the term “Rosiglitazone hydrobromide” refers to a Rosiglitazone hydrobromide salt, in which Rosiglitazone and hydrobromide are present in a molar ratio of about 1:1.

As used herein, the term “water content” refers to the content of water based upon the Loss on Drying method (the “LOD” method) as described in UPS 29-NF 24, official Aug. 1, 2006, Physical Test and Determinations, <731> LOSS ON DRYING or in Pharmacopeial Forum, Vol. 24, No. 1, p. 5438 (January-February 1998), the Karl Fisher assay for determining water content or thermogravimetric analysis (TGA). All percentages herein are by weight unless otherwise indicated. As used herein, the term “monohydrate” when used in reference to Rosiglitazone hydrobromide describes Rosiglitazone hydrobromide having water content of between about 3.9-4.1% w/w. As used herein, the term “hemi-hydrate” when used in reference to Rosiglitazone hydrobromide describes Rosiglitazone hydrobromide having a water content of about 1.8-2.1% w/w. As used herein, the term “quarter-hydrate” when used in reference to Rosiglitazone hydrobromide describes Rosiglitazone hydrobromide having a water content of about 0.7-1.0% w/w.

As used herein, the term chemical shift difference refers to the difference in chemical shift resonance between a reference chemical shift resonance and another chemical shift resonance in the same NMR spectrum. In the present patent application, the chemical shift differences were calculated by subtracting the lowest ppm resonance (reference chemical shift resonance) in the NMR spectrum of chemical shifts in the area of 100 to 180 ppm from another (observed) ppm resonance in the same NMR spectrum of chemical shifts in the area of 100 to 180 ppm. These chemical shift differences provide a measurement for a substance, for example Rosiglitazone hydrobromide, of the present invention that compensates for a phenomenon in NMR spectroscopy wherein, depending on the instrumentation and calibration method used, a shift in the SS-NMR “footprint” is observed. This shift in the SS-NMR “footprint”, having chemical shift resonances at a certain positions, is such that although the individual chemical shift resonances have altered, the distance between each chemical shift resonance and the next is retained.

In one embodiment, the present invention provides Rosiglitazone hydrobromide having a water content of between 1.0 and 4.1% by weight. Preferably, the Rosiglitazone hydrobromide is crystalline.

In another embodiment, the present invention provides crystalline Rosiglitazone hydrobromide quarter-hydrate.

In yet another embodiment, the persent invention provides crystalline Rosiglitazone hydrobromide hemihydrate.

Also provided in the present invention, is crystalline Rosiglitazone hydrobromide monohydrate.

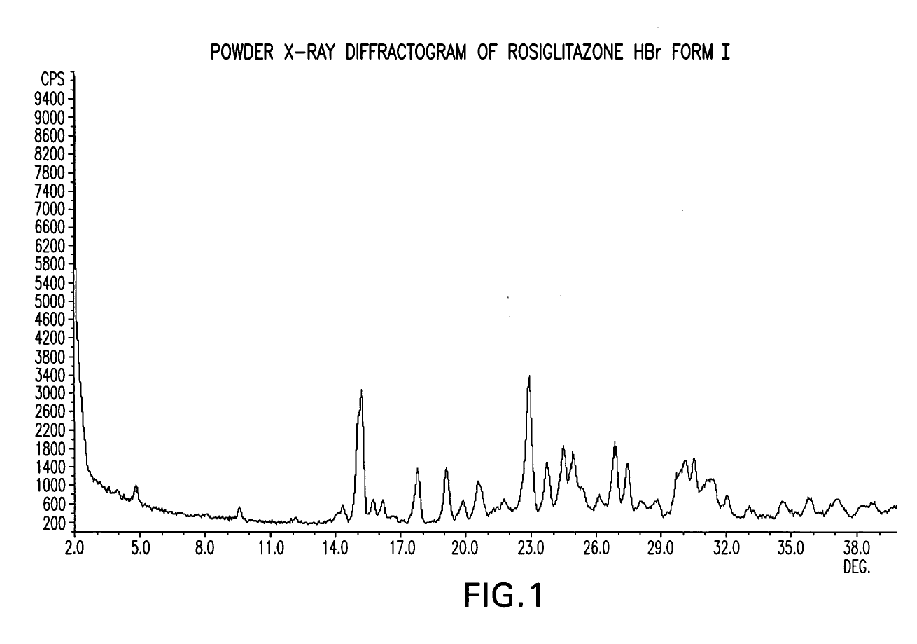

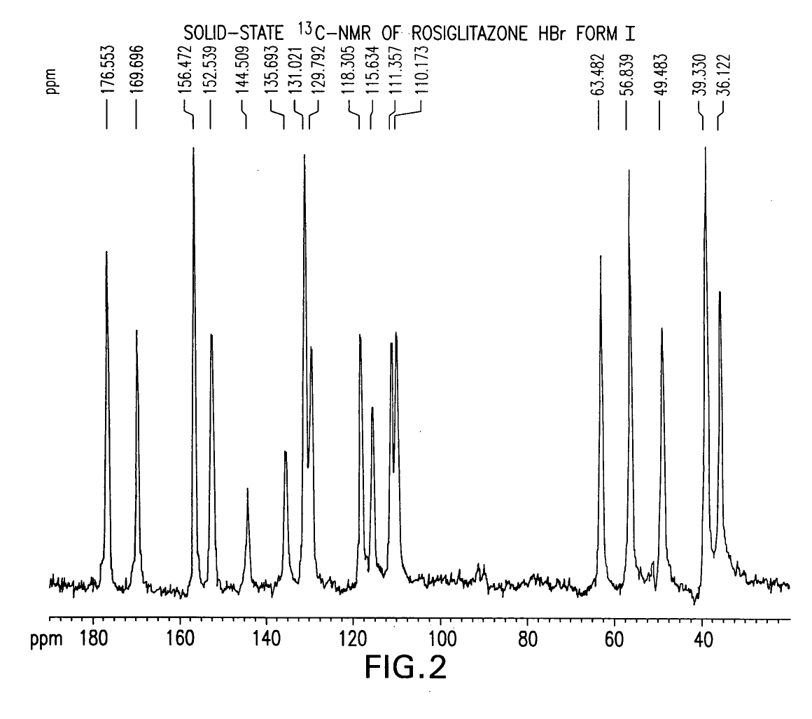

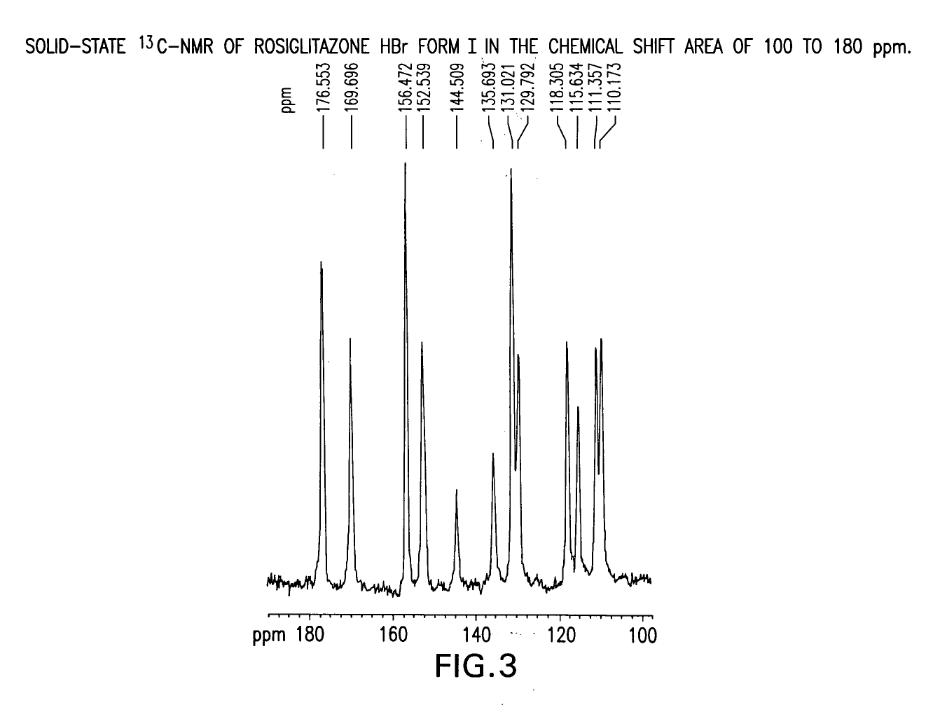

In one embodiment, the present invention provides a crystalline form of Rosiglitazone hydrobromide, designated Form I, characterized by at least one of data selected from the group consisting of: a powder XRD pattern having peaks at about 15.2, 17.8, 22.9 and 24.5±0.2 degrees two-theta; a solid-state ¹³C-NMR spectrum having chemical shift resonances at about 169.7, 156.5, 135.7, 118.3 and 115.6±0.2 ppm; a solid-state ¹³C NMR spectrum having chemical shift differences between the lowest ppm resonance in the chemical shift area of 100 to 180 ppm and another in the chemical shift area of 100 to 180 ppm of about 59.5, 46.3, 25.5, 8.1 and 5.4±0.1 ppm; an X-ray diffractogram substantially as depicted in FIG. 1; and solid-state ¹³C-NMR substantially as depicted in FIGS. 2 and 3.

Crystalline Rosiglitazone hydrobromide Form I may be further characterized by powder XRD pattern having peaks at about 19.2 and 26.9±0.2 degrees two-theta. Rosiglitazone hydrobromide Form I may have a weight loss, as measured by TGA, of between 0.7-4.1% by weight. It may have a water content, as measured by KF, of between 1.0-4.1% by weight.

When maintaining Rosiglitazone hydrobromide Form I at a relative humidity of about 100%, a monohydrate is obtained. When maintaining Rosiglitazone hydrobromide Form I at a relative humidity of about 50%, its water content is about 2%, which corresponds to a hemihydrate. When maintaining Rosiglitazone hydrobromide Form I at a relative humidity of about 0%, its water content is about 1%, which corresponds to a quarter-hydrate.

Crystalline Rosiglitazone hydrobromide Form I of the present invention preferably contains less than about 20%, preferably less than about 10%, more preferably less than about 5%, and most preferably less than about 1% by weight of the crystalline Rosiglitazone HBr described in WO 01/94344 or of crystalline Rosiglitazone form II, as judged by the presence of characteristic PXRD peaks for said crystalline Rosiglitazone HBr described in WO 01/94344, such as at 13.2, 13.4, 22.1, 23.4 degrees two-theta, or for form II.

Rosiglitazone hydrobromide Form I is provided as a solid material, in which Form I represents about 80%, preferably about 90%, more preferably about 95% and most preferably about 99% by weight of the solid material.

Rosiglitazone hydrobromide Form I of the present invention is stable under extreme heating conditions (80° C. for 30 minutes), as well as under high pressure and grinding conditions. Further, Rosiglitazone hydrobromide Form I of the present invention is also under extreme relative humidity conditions (100%). High stability to extreme relative humidity, heat, pressure or grinding conditions, which may be used during formulation processes or storage conditions is favorable in the pharmaceutical industry.

The present invention further encompasses a process for preparing the Rosiglitazone hydrobromide Form I by a process comprising; providing a mixture of Rosiglitazone and a solvent selected from the list consisting of methyl ethyl ketone, C₃₋₇ esters, water and mixture thereof; admixing the mixture with a source of hydrobromide; and cooling preferably to a temperature of about 0° C. to about 60° C. to obtain a precipitate. Preferably, the solvent is selected from the list consisting of methyl ethyl ketone, ethyl acetate, diethylcarbonate, water and mixtures thereof. Most preferably, the solvent is ethyl acetate. When ethyl acetate is used a solid precipitate and an oil are obtained, wherein the solid precipitate comprises Rosiglitazone form I.

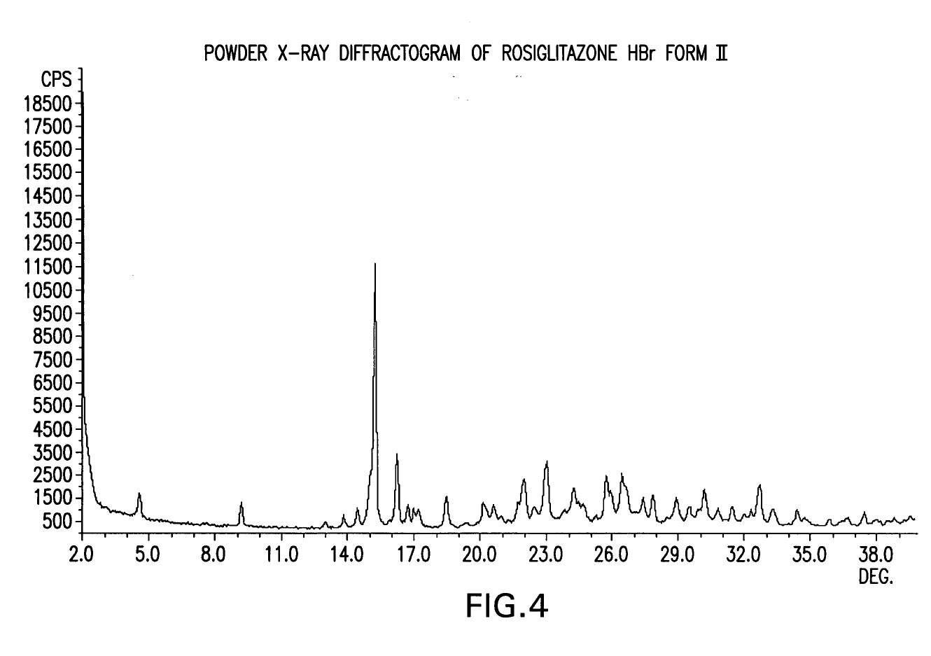

In another embodiment, the present invention provides a crystalline form of Rosiglitazone hydrobromide, designated Form II, characterized by a powder XRD pattern with peaks at about 4.6, 9.3, 15.3, 16.3 and 18.3±0.2 degrees two-theta. Crystalline Rosiglitazone Form II may be further characterized by powder XRD pattern with peaks at about 22.1, 25.8, 26.5 and 32.8±0.2 degrees two-theta. Crystalline Rosiglitazone Form I may have a powder XRD pattern substantially as depicted in FIG. 4. Rosiglitazone hydrobromide Form II may have a weight loss, as measured by TGA, of about 3.9% by weight. It may have a water content, as measured by KF of about 4.1% by weight. This corresponds to Rosiglitazone hydrobromide monohydrate.

Crystalline Rosiglitazone hydrobromide Form II of the present invention preferably contains less than about 20%, preferably less than about 10%, more preferably less than about 5%, and most preferably less than about 1% by weight of the crystalline Rosiglitazone HBr described in WO 01/94344 or of crystalline Rosiglitazone form I, as judged by the presence of characteristic PXRD peaks for said crystalline Rosiglitazon HBr described in WO 01/94344, such as at 13.2, 13.4, 22.1, 23.4 degrees two-theta, or for form I.

Rosiglitazone hydrobromide Form II is provided as a solid material, in which Form I represents about 80%, preferably about 90%, more preferably about 95% and most preferably about 99% by weight of the solid material.

The present invention further encompasses a process for preparing Rosiglitazone hydrobromide Form II by a process comprising; providing a mixture of Rosiglitazone and ethylacetate; admixing the mixture with a source of hydrobromide; cooling preferably to a temperature of about 0 to about 60° C. to obtain an oil; admixing the oil with a C₁-C₅ alcohol to obtain a precipitate; and recovering Rosiglitazone hydrobromide Form II.

Preferably, the C₁-C₅ alcohol is selected from methanol, ethanol, isopropanol, butanol and isobutanol. Most preferably, the C₁-C₅ alcohol is methanol.

Rosiglitazone used in the preparation of Forms I and II may be prepared by any method known in the art for example, according to the process described in International Patent Application No. WO94/05659 or EP 0306228.

Preferably, the solvent used in the preparation of Forms I and II is in a volume ratio in mL to gram Rosiglitazone of from about 5:1 to about 15:1, preferably about 10:1. The provided mixture of Rosiglitazone and the solvent is preferably obtained by raising the temperature sufficient to dissolve the Rosiglitazone, preferably to about reflux or a temperature between reflux temperature and about 20° C. below the reflux temperature.

The source of hydrogen bromide used in the preparation of Forms I and II may be an aqueous solution of hydrogen bromide, for example a 48% w/w solution of hydrogen bromide in water. Alternatively, the source of hydrogen bromide is a solution of hydrogen bromide in an appropriate solvent, optionally the reaction solvent. Alternatively, the hydrogen bromide may be added directly to the mixture of Rosiglitazone in the reaction solvent, preferably the reaction solvent is selected from the group consisting of methyl ethyl ketone, C₃₋₇ esters, preferably ethyl acetate and diethylcarbonate, water or mixture thereof. Most preferably, the reaction solvent is ethyl acetate. Another alternative source of hydrogen bromide may be hydrobromic acid (gas), or a base salt of hydrobromic acid for example ammonium bromide, or the hydrobromic acid salt of an amine (such as a C₁-C₉ amine), for example ethylamine or diethylamine. Preferably, the hydrogen bromide source is added drop-wise to the solution containing Rosiglitazone.

In the above processes of preparing Forms I or II cooling is preferably to a temperature of about 0° to about 50° C. or about 0° to about 60° C., more preferably, about 20° C. to about 25° C. Preferably cooling is for a period of about 12 hours to about 48 hours, more preferably for about 18 hours to about 48 hours.

The obtained Rosiglitazone hydrobromide Form I or From II may then recovered. Recovery can be carried out by filtration. The recovered Rosiglitazone can be dried. Drying can be carried out under a pressure of less than one atmosphere (reduced pressure), including a pressure of less than about 100 mmHg. Drying can also be carried out by heating, with or without reducing the pressure. Heating can be carried out to from room temperature to about 60° C., more preferably about 40° C. to about 60° C.

The present invention comprises a pharmaceutical composition comprising Rosiglitazone hydrobromide Form I or Form II of the present invention and at least one pharmaceutically acceptable excipient.

The present invention comprises a pharmaceutical composition comprising Rosiglitazone hydrobromide Form I or Form II made by the processes of the present invention, and at least one pharmaceutically acceptable excipient.

The present invention further encompasses a process for preparing a pharmaceutical formulation comprising combining Rosiglitazone hydrobromide Form I or Form II of the present invention with at least one pharmaceutically acceptable excipient.

The present invention further encompasses a process for preparing a pharmaceutical formulation comprising combining Rosiglitazone hydrobromide Form I or Form II made by the processes of the present invention, and at least one pharmaceutically acceptable excipient.

The present invention further encompasses the use of Rosiglitazone hydrobromide Form I or Form II of the present invention for the manufacture of a pharmaceutical composition.

The present invention further encompasses the use of Rosiglitazone hydrobromide Form I or Form II made by the processes of the invention, for the manufacture of a pharmaceutical composition.

Methods of administration of a pharmaceutical composition of the present invention may comprise administration in various preparations depending on the age, sex, and symptoms of the patient. The pharmaceutical compositions can be administered, for example, as tablets, pills, powders, liquids, suspensions, emulsions, granules, capsules, suppositories, injection preparations (solutions and suspensions), and the like. When the pharmaceutical composition comprises Rosiglitazone hydrobromide Form I or Form II, the liquid pharmaceutical composition is a suspension or emulsion, wherein Rosiglitazone hydrobromide Form I or Form II retains its crystalline form.

Pharmaceutical compositions of the present invention can optionally be mixed with other forms of Rosiglitazone hydrobromide and/or other active ingredients. In addition, pharmaceutical compositions of the present invention can contain inactive ingredients such as diluents, carriers, fillers, bulking agents, binders, disintegrants, disintegration inhibitors, absorption accelerators, wetting agents, lubricants, glidants, surface active agents, flavoring agents, and the like.

Diluents increase the bulk of a solid pharmaceutical composition and can make a pharmaceutical dosage form containing the composition easier for the patient and care giver to handle. Diluents for solid compositions include, for example, microcrystalline cellulose (e.g., Avicel®), microfine cellulose, lactose, starch, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol, or talc.

Carriers for use in the pharmaceutical compositions may include, but are not limited to, lactose, white sugar, sodium chloride, glucose, urea, starch, calcium carbonate, kaolin, crystalline cellulose, or silicic acid.

Binders help bind the active ingredient and other excipients together after compression. Binders for solid pharmaceutical compositions include for example acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, or starch.

Disintegrants can increase dissolution. Disintegrants include, for example, alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Disintegration inhibitors may include, but are not limited to, white sugar, stearin, coconut butter, hydrogenated oils, and the like.

Absorption accelerators may include, but are not limited to, quaternary ammonium base, sodium laurylsulfate, and the like.

Wetting agents may include, but are not limited to, glycerin, starch, and the like. Adsorbing agents may include, but are not limited to, starch, lactose, kaolin, bentonite, colloidal silicic acid, and the like.

A lubricant can be added to the composition to reduce adhesion and ease release of the product from a punch or dye during tableting. Lubricants include for example magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Glidants can be added to improve the flowability of non-compacted solid composition and improve the accuracy of dosing. Excipients that can function as glidants include for example colloidal silicon dioxide, magnesium trisilicate, powdered cellulose, starch, talc and tribasic calcium phosphate.

Flavoring agents and flavor enhancers make the dosage form more palatable to the patient. Common flavoring agents and flavor enhancers for pharmaceutical products that can be included in the composition of the present invention include for example maltol, vanillin, ethyl vanillin, menthol, citric acid, fumaric acid, ethyl maltol, and tartaric acid.

Tablets can be further coated with commonly known coating materials such as sugar coated tablets, gelatin film coated tablets, tablets coated with enteric coatings, tablets coated with films, double layered tablets, and multi-layered tablets. Capsules can be coated with shell made, for example, from gelatin and optionally contain a plasticizer such as glycerin and sorbitol, and an opacifying agent or colorant.

Solid and liquid compositions can also be dyed using any pharmaceutically acceptable colorant to improve their appearance and/or facilitate patient identification of the product and unit dosage level.

In liquid pharmaceutical compositions of the present invention, the Rosiglitazone hydrobromide Form I or Form II of the present invention is suspended, while retaining its crystalline form, together with and any other solid ingredients, which may be dissolved or suspended, in a liquid carrier, such as water, vegetable oil, alcohol, polyethylene glycol, propylene glycol or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention can also contain viscosity enhancing agents to improve the mouth-feel of the product and/or coat the lining of the gastrointestinal tract. Such agents include for example acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar can be added to improve the taste.

Preservatives and chelating agents such as alcohol, sodium benzoate, butylated hydroxy toluene, butylated hydroxyanisole and ethylenediamine tetraacetic acid can be added at safe levels to improve storage stability.

A liquid pharmaceutical composition according to the present invention can also contain a buffer such as guconic acid, lactic acid, citric acid or acetic acid, sodium guconate, sodium lactate, sodium citrate or sodium acetate.

Selection of excipients and the amounts to use can be readily determined by an experienced formulation scientist in view of standard procedures and reference works known in the art.

A composition for tableting or capsule filing can be prepared by wet granulation. In wet granulation some or all of the active ingredients and excipients in powder form are blended and then further mixed in the presence of a liquid, typically water, which causes the powders to clump up into granules. The granulate is screened and/or milled, dried and then screened and/or milled to the desired particle size. The granulate can then be tableted or other excipients can be added prior to tableting, such as a glidant and/or a lubricant.

A tableting composition can be prepared conventionally by dry blending. For instance, the blended composition of the actives and excipients can be compacted into a slug or a sheet and then comminuted into compacted granules. The compacted granules can be compressed subsequently into a tablet.

As an alternative to dry granulation, a blended composition can be compressed directly into a compacted dosage form using direct compression techniques. Direct compression produces a more uniform tablet without granules. Excipients that are particularly well-suited to direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention can comprise any of the aforementioned blends and granulates that were described with reference to tableting, only they are not subjected to a final tableting step.

When shaping the pharmaceutical composition into pill form, any commonly known excipient used in the art can be used. For example, carriers include, but are not limited to, lactose, starch, coconut butter, hardened vegetable oils, kaolin, talc, and the like. Binders used include, but are not limited to, gum arabic powder, tragacanth gum powder, gelatin, ethanol, and the like. Disintegrating agents used include, but are not limited to, agar, laminalia, and the like.

For the purpose of shaping the pharmaceutical composition in the form of suppositories, any commonly known excipient used in the art can be used. For example, excipients include, but are not limited to, polyethylene glycols, coconut butter, higher alcohols, esters of higher alcohols, gelatin, semisynthesized glycerides, and the like.

When preparing injectable pharmaceutical compositions, solutions and suspensions are sterilized and are preferably made isotonic to blood. Injection preparations may use carriers commonly known in the art. For example, carriers for injectable preparations include, but are not limited to, water, ethyl alcohol, propylene glycol, ethoxylated isostearyl alcohol, polyoxylated isostearyl alcohol, and fatty acid esters of polyoxyethylene sorbitan. One of ordinary skill in the art can easily determine with little or no experimentation the amount of sodium chloride, glucose, or glycerin necessary to make the injectable preparation isotonic. Additional ingredients, such as dissolving agents, buffer agents, and analgesic agents may be added.

The amount of Rosiglitazone hydrobromide Form I or Form II of the present invention contained in a pharmaceutical composition according to the present invention is not specifically restricted; however, the dose should be sufficient to treat, ameliorate, or reduce the condition.

Having described the invention with reference to certain preferred embodiments, other embodiments will become apparent to one skilled in the art from consideration of the specification. The disclosures of the references referred to in this patent application are incorporated herein by reference. The invention is further defined by reference to the following examples describing in detail the process and compositions of the invention. It will be apparent to those skilled in the art that many modifications, both to materials and methods, may be practiced without departing from the scope of the invention.

### EXAMPLES

### Instruments

### XRD

Powder X-ray diffraction (“XRD”) analysis can be carried out using any XRD powder diffractometer commonly used in the industry. The Rosiglitazone hydrobromide Form I samples of this invention were run in a SCINTAG powder X-ray diffractometer model X'TRA equipped with a solid-state detector. Copper radiation of λ=1.5418. The sample can be introduced using a round standard aluminum sample holder with round zero background quartz plate in the bottom and is scanned by a continuous scan at a rate of 3° per minute. All peak positions reported are within ±0.2 degrees two theta.

### TGA Analysis

TGA analysis was preformed using Mettler 3M with Mettler TG 50 thermobalance.

The weight of the samples was about 10 mg; the samples were scanned at a rate of 10° C./min from 25° C. to 200 or 250° C. The oven was constantly purged with nitrogen gas at a flow rate of 40 ml/min. Standard alumina crucibles covered by lids with 1 hole were used.

### Water Content

Water content was determined by Karl Fisher analysis using Mettler Toledo DL 38 Karl Fisher Titrator.

### Solid-State ¹³C NMR Spectroscopy

The cp/mas ¹³C NMR investigations were preformed at 125.76 MHz at ambient temperature on a Bruker DMX-500 digital FT-NMR spectrometer equipped with a BL-4 cp/mas probehead and High Resolution/High Performance (HPHP) 1H and X-channel preamplifiers for solids. Samples were placed in 4 mm zirconia rotors fitted with ‘Kel-F’ plastic caps, and spun with dry air at 5.0 kHz.

### Preparation and Stability of Rosiglitazone Hydrobromic Acid Form I

### Example 1

A 100 ml flask was charged with Rosiglitazone (3 g) and methylethylketone (30 ml). The suspension was heated to reflux and then aqueous Hydrobromic acid 48% was added (0.95 ml) drop wise to obtain a clear solution, stirred for an additional 10 minutes and cooled to room temperature while stirring for 2 days. The resulting solid was filtered under reduced pressure, and dried at 50° C. under reduced pressure to give 3.05 g (82.9%) of a white solid. The resulting solid was analyzed by XRD to yield Rosiglitazone hydrobromic acid Form I.

### Example 2

A 100 ml flask was charged with Rosiglitazone (3 g) and water (30 ml). The suspension was heated to reflux and then aqueous Hydrobromic acid 48% was added (0.95 ml) drop wise to obtain a clear solution, stirred for an additional 10 minutes and cooled to room temperature while stirring for 19 hours. The resulting solid was filtered under reduced pressure and dried at 50° C. under reduced pressure to give 2.67 g (71.6%) of a white solid. The resulting solid was analyzed by XRD to yield Rosiglitazone hydrobromic acid Form I. (See FIG. 1)

### Example 3

A 100 ml flask was charged with Rosiglitazone (3 g) and ethyl acetate (30 ml). The suspension was heated to reflux and then aqueous Hydrobromic acid 48% was added (0.95 ml) drop wise to obtain an oily suspension, stirred for an additional 10 minutes and cooled to room temperature while stirring for 19 hours to obtain part oil and part solid. The mixture containing the solid was then decanted out, filtered under reduced pressure and dried at 50° C. to give 0.20 g (5.4%) of a white solid. The resulting solid was analyzed by XRD to yield Rosiglitazone hydrobromic acid Form I.

### Example 4

A 100 ml flask was charged with Rosiglitazone (3 g) and Diethylcarbonate (30 ml). The suspension was heated to reflux and then aqueous Hydrobromic acid 48% was added (0.95 ml) drop wise to obtain an oily suspension, stirred for an additional 10 minutes and cooled to room temperature while stirring for 19 hours to remain an oily mixture. The solvent was removed by decantation and methanol was added. The suspension was stirred at room temperature for 20 hours. The resulting solid was filtered under reduced pressure, and dried at 50° C. under reduced pressure to give 2.49 g (67.3%) of a white solid. The resulting solid was analyzed by XRD to yield Rosiglitazone hydrobromic acid Form I.

### Example 5

Form I was subjected to various conditions to determine crystalline stabitlity. Particularly, Form I was pressed by laboratory press, ground by mortal and pestle, and heated in a laboratory oven, for the time periods as indicated in the table 1. X-ray powder diffraction of the crystalline material as obtained after these conditions was measured.

TABLE 1

| Pressure | | | Grinding | | | Heating | | |
|---|---|---|---|---|---|---|---|---|
| Pressure | Time | Resulting | Grinding | Time | Resulting | Temperature | Time | Resulting |
| (ton) | (min) | form | (wet/dry) | (min) | form | (° C.) | (min) | form |
| 2 | 1 | I | dry | 1 | I | 80 | 30 | I |

### Example 6

Form I was stored at room temperature for 7 days at levels of relative humidity, as indicated in table 2. An X-ray powder diffraction was measured for each crystalline material after such storage conditions. The weight loss on drying as measured by TGA and the water content according to the KF analysis were also determined.

TABLE 2

| % RH | TGA | KF | Form |
|---|---|---|---|
| As is | 4.1 | 4.1 | I |
| 0 | 0.7 | 1.0 | I |
| 100 | 3.9 | 4.1 | I |

### Example 7

Solid pharmaceutical compositions of Form I and the following excipients were compacted into a dosage form like a tablet: lactose monohydrate, sucrose and avicel. The polymorphic stability of Rosiglitazone form I in such tablet was examined and determined to be stable.

### Preparation of Rosiglitazone Hydrobromic Acid Form II

### Example 8

The oil obtained and decanted out in Example 3 was mixed with methanol. The mixture was stirred for 20 hours at room temperature. The resulting solid was filtered under reduced pressure, and dried at 50° C. under reduced pressure to give 0.77 g (20.9%) of a white solid. The resulting solid was analyzed by XRD to yield Rosiglitazone hydrobromic acid Form II.

What is claimed:

1. Rosiglitazone hydrobromide having a water content of between 1 and 4.1% by weight.

2. The Rosiglitazone hydrobromide of claim 1, wherein the Rosiglitazone hydrobromide is crystalline.

3. The Rosiglitazone hydrobromide of claim 1, wherein the Rosiglitazone hydrobromide is a monohydrate.

4. The Rosiglitazone hydrobromide of claim 1, wherein the Rosiglitazone hydrobromide is a hemihydrate.

5. The Rosiglitazone hydrobromide of claim 1, wherein the Rosiglitazone hydrobromide is a quarter-hydrate.

6. A Rosiglitazone hydrobromide crystalline form, characterized by at least one of data selected from the group consisting of: a powder XRD pattern having peaks at about 15.2, 17.8, 22.9 and 24.5±0.2 degrees two-theta; a solid-state ¹³C-NMR spectrum having chemical shift resonances at about 169.7, 156.5, 135.7, 118.3 and 115.6±0.2 ppm; a solid-state ¹³C NMR spectrum having chemical shift differences between the lowest ppm resonance in the chemical shift area of 100 to 180 ppm and another in the chemical shift area of 100 to 180 ppm of about 59.5, 46.3, 25.5, 8.1 and 5.4±0.1 ppm; an X-ray diffractogram substantially as depicted in FIG. 1; and a solid-state ¹³C-NMR substantially as depicted in FIG. 2 or 3.

7. The Rosiglitazone hydrobromide crystalline form of claim 6, characterized by a powder XRD pattern having peaks at about 15.2, 17.8, 22.9 and 24.5±0.2 degrees two-theta.

8. The Rosiglitazone hydrobromide crystalline form of claim 7, further characterized by powder XRD pattern having peaks at about 19.2 and 26.9±0.2 degrees two-theta.

9. The Rosiglitazone hydrobromide crystalline form of claim 6, characterized by an X-ray diffractogram substantially as depicted in FIG. 1.

10. The Rosiglitazone hydrobromide crystalline form of claim 6, characterized by a solid-state ¹³C-NMR spectrum having chemical shift resonances at about 169.7, 156.5, 135.7, 118.3 and 115.6±0.2 ppm.

11. The Rosiglitazone hydrobromide crystalline form of claim 6, characterized by a solid-state ¹³C NMR spectrum having chemical shift differences between the lowest ppm resonance in the chemical shift area of 100 to 180 ppm and another in the chemical shift area of 100 to 180 ppm of about 59.5, 46.3, 25.5, 8.1 and 5.4±0.1 ppm.

12. The Rosiglitazone hydrobromide crystalline form of claim 6, characterized by a solid-state ¹³C-NMR substantially as depicted in FIG. 2 or 3.

13. The Rosiglitazone hydrobromide crystalline form of claim 6, having a weight loss, as measured by TGA, of between about 0.7-4.1% by weight.

14. The Rosiglitazone hydrobromide crystalline form of claim 6, having a water content, as measured by KF, of between about 1.0-4.1% by weight.

15. The Rosiglitazone hydrobromide crystalline form of claim 6, wherein the Rosiglitazone hydrobromide crystalline form is a monohydrate.

16. The Rosiglitazone hydrobromide crystalline form of claim 6, wherein the Rosiglitazone hydrobromide crystalline form is a hemihydrate.

17. The Rosiglitazone hydrobromide crystalline form of claim 6, wherein the Rosiglitazone hydrobromide crystalline form is a quarter-hydrate.

18. A process for preparing the crystalline Rosiglitazone hydrobromide of claim 6 comprising:
a. providing a mixture of Rosiglitazone and a solvent selected from the list consisting of methyl ethyl ketone, ethyl acetate, diethylcarbonate and water or mixture thereof;
b. admixing the mixture with a source of hydrobromide; and
c. cooling the mixture to obtain a precipitate.

19. The process of claim 18, wherein the solvent to Rosiglitazone ratio in step a) is in a volume ratio of from about 5:1 to about 15:1 (mL to gram Rosiglitazone).

20. The process of claim 19, wherein the ration is about 10:1.

21. The process of claim 18, wherein the mixture is provided by dissolving Rosiglitazone in the solvent at a temperature sufficient to dissolve the Rosiglitazone.

22. The process of claim 21, wherein the temperature is about reflux.

23. The process of claim 18, wherein the source of hydrogen bromide is selected from an aqueous solution of hydrogen bromide, a solution of hydrogen bromide in the solvent, hydrobromic acid (gas), or a base salt of hydrobromic acid, or hydrobromic acid salt of an amine.

24. The process of claim 18, wherein the hydrogen bromide source is added drop-wise to the solution containing Rosiglitazone.

25. The process of claim 18, wherein the hydrogen bromide source is added directly to the mixture of Rosiglitazone in the solvent.

26. The process of claim 18, wherein the cooling in step c) is to a temperature of about 0° C. to about 60° C.

27. The process of claim 26, wherein the cooling in step c) is to a temperature of about 20° C. to about 25° C.

28. The process of claim 18, wherein cooling is for a period of about 12 hours to about 48 hours.

29. A Rosiglitazone hydrobromide crystalline form characterized by a powder XRD pattern with peaks at about 4.6, 9.3, 15.3, 16.3 and 18.3±0.2 degrees two-theta.

30. The Rosiglitazone hydrobromide crystalline form of claim 29, further characterized by powder XRD pattern having peaks at about 22.1, 25.8, 26.5 and 32.8±0.2 degrees two-theta.

31. The Rosiglitazone hydrobromide crystalline form of claim 29 characterized by an X-ray diffractogram substantially as depicted in FIG. 3.

32. The Rosiglitazone hydrobromide crystalline form of claim 29, having a weight loss, as measured by TGA, of about 3.9% by weight.

33. The Rosiglitazone hydrobromide crystalline form of claim 29, having a water content, as measured by KF of about 4.1% by weight.

34. The Rosiglitazone hydrobromide crystalline form of claim 29, wherein the Rosiglitazone hydrobromide crystalline form is a hydrate.

35. The Rosiglitazone hydrobromide crystalline form of claim 29, wherein the crystalline form is a monohydrate.

36. A process for preparing the Rosiglitazone hydrobromide crystalline form of claim 29, comprising:
a. providing a mixture of Rosiglitazone and ethylacetate;
b. admixing the mixture with a source of hydrobromide;
c. cooling the mixture to obtain an oil;
d. admixing said oil with C₁-C₅ alcohol to obtain a precipitate.

37. The process of claim 36, wherein the solvent to Rosiglitazone ratio in step a) is in a volume ratio of from about 5:1 to about 15:1 (mL to gram Rosiglitazone).

38. The process of claim 37, wherein the ration is about 10:1.

39. The process of claim 36, wherein the mixture is provided by dissolving Rosiglitazone in the solvent at a temperature sufficient to dissolve the Rosiglitazone.

40. The process of claim 39, wherein the temperature is about reflux.

41. The process of claim 36, wherein the source of hydrogen bromide is selected from an aqueous solution of hydrogen bromide, a solution of hydrogen bromide in the solvent, hydrobromic acid (gas), or a base salt of hydrobromic acid, or hydrobromic acid salt of an amine.

42. The process of claim 36, wherein the hydrogen bromide source is added drop-wise to the solution containing Rosiglitazone.

43. The process of claim 36, wherein the hydrogen bromide source is added directly to the mixture of Rosiglitazone in the solvent.

44. The process of claim 36, wherein the cooling in step c) is to a temperature of about 0° C. to about 60° C.

45. The process of claim 44, wherein the cooling in step c) is to a temperature of about 20° C. to about 25° C.

46. The process of claim 36, wherein cooling is for a period of about 12 hours to about 48 hours.

47. The process of claim 36, wherein the C₁-C₅ alcohol is methanol.

48. A pharmaceutical composition comprising the Rosiglitazone hydrobromide crystalline form of claim 1, 6 or of claim 29 and at least one pharmaceutically acceptable excipient.

49. A pharmaceutical composition comprising the Rosiglitazone hydrobromide crystalline forms made by the process of claim 18 or claim 36, and at least one pharmaceutically acceptable excipient.

50. A process for preparing a pharmaceutical formulation comprising combining the Rosiglitazone hydrobromide crystalline form of claim 1, 6 or of claim 29 with at least one pharmaceutically acceptable excipient.

51. A process for preparing a pharmaceutical formulation comprising combining the Rosiglitazone hydrobromide crystalline forms made by the process of claim 18 or claim 36, and at least one pharmaceutically acceptable excipient.
